(19) United States
(12) Patent Application Publication
(10) Pub. No.: US 2025/0197346 A1
(43) Pub. Date: Jun. 19, 2025

(54) COMPOUND FOR OPTICAL ABSORBENT

(71) Applicant: LMS Co., Ltd., Pyeongtaek-si (KR)

(72) Inventors: Joon Ho JUNG, Pyeongtaek-si (KR); Seung Chul LEE, Pyeongtaek-si (KR); Hee Kyeong KIM, Pyeongtaek-si (KR)

(21) Appl. No.: 18/947,417

(22) Filed: Nov. 14, 2024

(30) Foreign Application Priority Data
Dec. 14, 2023 (KR) 10-2023-0181998

(51) Int. Cl. C07C 251/20; C07C 311/09; C08K 5/435; C09D 5/00; C09D 7/63; G02B 1/04; G02B 5/20

(52) U.S. Cl. CPC C07C 251/20; C07C 311/09; C08K 5/435; C09D 5/006; C09D 7/63; G02B 1/04; C07B 2200/05; G02B 5/208

(57) ABSTRACT

Provided are optical absorbent compounds and their uses. The compounds have excellent heat resistance and can stably maintain light absorption characteristics even under high temperature conditions or high temperature and high humidity conditions. A resin film secures desired optical properties by applying the compounds. The compounds can be used as an optical absorbent for an optical filter.

### CROSS-REFERENCE TO RELATED APPLICATION(S)

This application is based on and claims priority under 35 U.S.C. 119 to Korean Patent Application No. 10-2023-0181998, filed on Dec. 14, 2023, in the Korean Intellectual Property Office, the disclosure of which is herein incorporated by reference in its entirety.

### TECHNICAL FIELD

This specification relates to optical absorbent compounds and their uses.

### BACKGROUND

A compound that can be used as an optical absorbent, for example, a compound capable of absorbing light in the infrared region can be applied to various applications. Because an image capturing device or an infrared sensor using a CCD (Charge-Coupled Device) or a CMOS (Complementary Metal-Oxide-Semiconductor) image sensor, for example, includes a silicon photodiode having sensitivity to the near-infrared region, the optical absorbent may be used for them.

As for the optical absorbents include, for example, phthalocyanine-based, cyanine-based, and metal dithiol complex-based, squarylium-based and dimmonium salt-based compounds are known. The phthalocyanine-based compounds are known as near-infrared compounds, but they have the problem of high absorption in the visible light region. In addition, cyanine-based compounds have a problem because the near-infrared region that can be absorbed by a single compound is narrow, they must be used in combination with other compounds. Additionally, because metal dithiol complex-based compounds have low solubility, additional dispersion equipment is required when they are applied to films, and thus, they are difficult to be applied for applications requiring high permeability.

In addition, squarylium-based compounds are known as compounds with excellent heat resistance. However, squarylium-based compounds have difficulty absorbing light in the long wavelength range of 900 nm or longer. Additionally, iminium or dimmonium-based compounds are known to be compounds that can absorb light with a wavelength of 900 nm or longer.

However, iminium or dimmonium-based compounds have a problem of losing light absorption characteristics in high temperature and/or high humidity environments due to low thermal stability.

To resolve the difficulties as mentioned above, the present invention may disclose compounds and their uses which can provide the compounds having excellent heat resistance and capable of stably maintaining light absorption characteristics even when maintained under high temperature conditions or high temperature and high humidity conditions. Moreover, the present specification can also provide a resin film that secures desired optical properties by applying the above-mentioned compounds.

### SUMMARY

The present specification is to disclose compounds and their uses.

The object of the present specification is to disclose the compounds that have excellent heat resistance and can stably maintain light absorption characteristics even when high temperature conditions or high temperature and high humidity conditions are maintained.

Another object of the present specification is to provide a resin film to secure desired optical properties by applying the compounds.

Yet another object of the present specification is to disclose the use of the compounds.

According to an embodiment of the invention, there is provided that an optical absorbent composition comprises a compound represented by Formula 1:

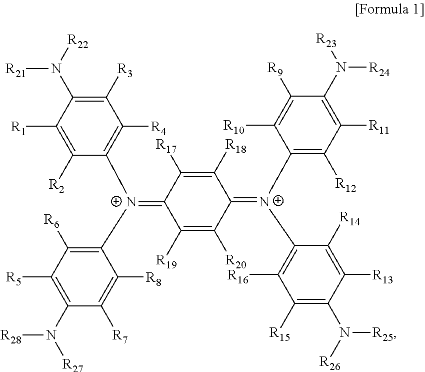

where R₁ to R₂₈ are each independently hydrogen, deuterium, an alkyl group, an alkenyl group, an alkynyl group, an alkoxy group, an aryl group, an aryloxy group, an arylamino group, an alkylamino group, a heteroaryl group, an alkylsilyl group, an arylsilyl group, an amino group, a nitro group, a nitrile group, a hydroxy group, or cyano group; and at least one of R₁ to R₂₈ is deuterium.

In an embodiment, a deuterium substitution rate of the compound is 10% or higher.

In an embodiment, the compound exhibits an absorption maximum within a wavelength range of about 690 nm to 1,200 nm.

In an embodiment, a transmittance of the compound at the absorption maximum is about 50% or less.

In an embodiment, a molar mass of the compound is in a range of about 900 g/mol to 3,000 g/mol.

In an embodiment, a 5% pyrolysis temperature of the compound is at least about 285° C.

In an embodiment, the compound further comprises an anion.

In an embodiment, a resin film comprises a resin component and the compound.

In an embodiment, the resin component for the resin film further comprises at least one or more selected from a group consisting of a cyclic olefin resin, a polyarylate resin, a polyester resin, a polysulfone resin, a polyether sulfone resin, a polyparaphenylene resin, a polyarylene ether phosphine oxide resin, a polyimide resin, a polyetherimide resin, a polyamidoimide resin, an acrylic resin, a polycarbonate resin, a polyethylene naphthalate resin, and a silicone resin.

In an embodiment, the resin component for the resin film contains a cyclic olefin resin.

In an embodiment, the compound for the resin film further comprises an anion.

In an embodiment, the compound is comprised in the resin film an amount of 0.001 to 10 parts by weight relative to 100 parts by weight of the resin component.

In an embodiment, the resin film exhibits an absorption maximum within a wavelength range of about 690 nm to 1,200 nm.

In an embodiment, an absolute value of AA for the resin film is about 80% or less in Equation 1:

$$Δ A = 100 \times ( {A}_{f} - {A}_{i} ) / {A}_{i} , \qquad [ Equation 1 ]$$

where A_{f} is a transmittance at a wavelength of the absorption maximum of the resin film which is after being maintained at 85° C. and 85% relative humidity for 120 hours and Aᵢ is a transmittance at a wavelength of the absorption maximum of the resin film which is before being maintained at 85° C. and 85% relative humidity for 120 hours.

In an embodiment, an absolute value of AX for the resin film is about 10% or less in Equation 2:

$$Δλ = 100 \times ( {λ}_{f} - {λ}_{i} ) / {λ}_{i} , \qquad [ Equation 2 ]$$

where λ_{f} is a wavelength of the absorption maximum of the resin film which is after being maintained at 85° C. and 85% relative humidity for 120 hours and λᵢ is a wavelength of the absorption maximum of the resin film which is before being maintained at 85° C. and 85% relative humidity for 120 hours.

In an embodiment, the wavelength of the absorption maximum of the resin film exists within a wavelength range of about 690 nm to 1,200 nm.

In an embodiment, an optical filter comprises a substrate and the resin film formed on one or both sides of the substrate.

In an embodiment, an image capturing device comprises the optical filter.

In an embodiment, an infrared sensor comprises the resin film.

### BRIEF DESCRIPTION OF THE DRAWINGS

FIGS. 1 to 3 are drawings showing exemplary structures of optical filters of the present invention.

FIGS. 4 to 7 are spectra showing the light absorption characteristics of resin films containing compounds of Embodiments or Comparative Examples before and after high-temperature and high-humidity evaluation.

### DETAILED DESCRIPTION

For those physical properties mentioned in the present invention where the result of measuring temperature may affect, it is measured at room temperature unless otherwise specified. The term “room temperature” used in the present invention refers to a natural temperature that is intentionally not heated or not reduced, for example, it means any temperature within the range of 10° C. to 30° C., a temperature of about 23° C. or about 25° C. In addition, in the present specification, the unit of temperature is Celsius (° C.) unless otherwise specified.

In the case of a physical property in which the measured humidity affects the result, the physical property is a physical property measured at natural humidity that is not specifically controlled at the room temperature and/or atmosphere pressure. The term “atmospheric pressure” is a natural pressure that is intentionally not pressurized or depressurized. It usually indicates that about 1 atmosphere of atmospheric pressure having the value of about 700 mmHg to 800 mmHg.

Among the physical properties mentioned in the present specification, in cases where humidity affects the results, the relevant physical properties are those measured at standard humidity unless otherwise specified. Humidity in a standard state means any humidity in the range of 40% to 60% relative humidity, for example, about 40% or 60% relative humidity.

In the case where the optical characteristic (for example, refractive index) mentioned in the present specification is a characteristic that varies depending on the wavelength, the optical characteristic is a characteristic for light with a wavelength of 520 nm unless specifically specified otherwise.

In the present specification, unless otherwise specified, the term “transmittance” or “absorption rate” refers to the actual transmittance (actual transmittance) or actual absorption rate (actual absorption rate) confirmed within a specific wavelength or wavelength range of a certain region. It is the transmittance or absorption rate with respect to an incident angle of 0°.

The term “average transmittance” or “average absorption rate”, unless specifically specified otherwise, is the result obtained from the arithmetic mean of the transmittance or absorption rate measured after measuring the transmittance or absorption rate of each wavelength while increasing the wavelength by 1 nm starting from the shortest wavelength within a given wavelength range. For example, the average transmittance or average absorption rate within the wavelength range of 350 nm to 360 nm may mean the arithmetic mean of the transmittance or absorption measured at a wavelength of 350 nm, 351 nm, 352 nm, 353 nm, 354 nm, 355 nm, 356 nm, 357 nm, 358 nm, 359 nm, and 360 nm.

The term “maximum transmittance” or “maximum absorption rate” refers to the maximum transmittance or maximum absorption rate when the transmittance or absorption rate of each wavelength is measured while increasing the wavelength by 1 nm starting from the shortest wavelength within a certain wavelength range. For example, the maximum transmittance or maximum absorption rate within the wavelength range of 350 nm to 360 nm may mean the highest transmittance or absorption rate among the transmittances measured at a wavelength of 350 nm, 351 nm, 352 nm, 353 nm, 354 nm, 355 nm, 356 nm, 357 nm, 358 nm, 359 nm, and 360 nm.

The term “minimum transmittance” or “minimum absorption rate” refers to the minimum transmittance or absorption rate when the transmittance or absorption rate of each wavelength is measured while increasing the wavelength by 1 nm starting from the shortest wavelength within a certain wavelength range. For example, the minimum transmittance or minimum absorption rate within the wavelength range of 350 nm to 360 nm may mean the lowest transmittance or absorption rate among those measured at a wavelength of 350 nm, 351 nm, 352 nm, 353 nm, 354 nm, 355 nm, 356 nm, 357 nm, 358 nm, 359 nm, and 360 nm.

The term “incident angle” is an angle measured with respect to the normal line of the surface to be evaluated. For example, the transmittance at an incident angle of 0° of an optical filter means the transmittance of light incident in a direction substantially parallel to the normal line of the surface of the optical filter. This definition of the incident angle applies equally to other characteristics such as transmittance and absorption rate.

In the specification, the term “alkyl group” means an alkyl group having 1 to 30 carbon numbers, 1 to 24 carbon numbers, 1 to 20 carbon numbers, 1 to 16 carbon numbers, 1 to 12 carbon numbers, 1 to 8 carbon numbers, or 1 to 4 carbon numbers. The alkyl group can be straight-chain, branched-chain or cyclic. The alkyl group may optionally be substituted with one or more substituents. These contents are applied to all alkyl groups mentioned in the specification unless otherwise specified.

In the specification, the term “alkenyl group” means an alkenyl group having 2 to 30 carbon numbers, 2 to 24 carbon numbers, 2 to 20 carbon numbers, 2 to 16 carbon numbers, 2 to 12 carbon numbers, 2 to 8 carbon numbers, or 2 to 4 carbon numbers. The alkenyl group can be straight-chain, branched-chain or cyclic. The alkenyl group may be optionally substituted with one or more substituents. These contents apply to all alkenyl groups mentioned in the specification unless otherwise specified.

In the specification, the term “alkynyl group” means an alkynyl group having 2 to 20 carbon numbers, 2 to 16 carbon numbers, 2 to 12 carbon numbers, 2 to 8 carbon numbers, or 2 to 4 carbon numbers. The alkynyl group can be straight-chain, branched-chain or cyclic. The alkynyl group may be optionally substituted with one or more substituents. These contents apply to all alkynyl groups mentioned in the specification unless otherwise specified.

In the specification, the term “alkoxy group” means an alkoxy group having 1 to 30 carbon numbers, 1 to 24 carbon numbers, 1 to 20 carbon numbers, 1 to 16 carbon numbers, 1 to 12 carbon numbers, 1 to 8 carbon numbers, or 1 to 4 carbon numbers. The alkoxy group can be straight-chain, branched-chain or cyclic. The alkoxy group may be optionally substituted with one or more substituents. These contents are applied to all alkoxy groups mentioned in the specification unless otherwise specified.

In the specification, the term “aryl group” means a monovalent moiety derived from an aromatic hydrocarbon, and the aryl group may be an aryl group having 6 to 48 carbon numbers, 6 to 42 carbon numbers, 6 to 36 carbon numbers, 6 to 30 carbon numbers, 6 to 24 carbon numbers, 6 to 18 carbon numbers, or 6 to 12 carbon numbers. It may be, for example, a phenyl group, a tolyl group, a xylyl group, or a naphthyl group. The aryl group may optionally be substituted by one or more substituents. These contents are applied to all aryl groups mentioned in the specification unless otherwise specified.

Additionally, in the present specification, the aryl group may be, for example, a heteroaryl group, and the heteroaryl group is a structure that includes a heteroatom, for example, O, N, or S, in the ring structure of the aryl group other than a carbon atom. The heteroaryl group may be optionally substituted with one or more substituents. These contents are applied to all heteroaryl groups mentioned in the specification unless otherwise specified.

This specification discloses compounds. The compound may be an optical absorbent. The term “optical absorbent” refers to a compound capable of absorbing light in any wavelength range.

The compound may be a compound represented by the following Formula 1.

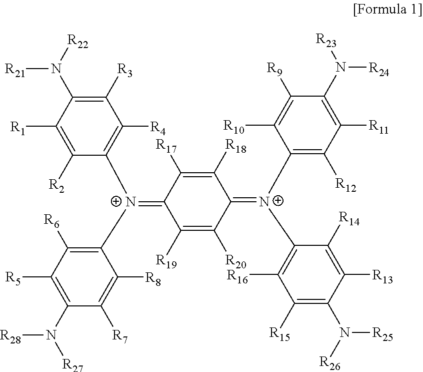

In Formula 1, R₁ to R₂₈ may be each independently hydrogen, deuterium, an alkyl group, an alkenyl group, an alkynyl group, an alkoxy group, an aryl group, an aryloxy group, an arylamino group, an alkylamino group, a heteroaryl group, a silyl group, an amino group, a nitro group, a nitrile group, a hydroxy group, or a cyano group.

The silyl group may be a silyl group (alkylsilyl group) where at least one alkyl group is bonded to silicon or a silyl group (arylsilyl group) where at least one aryl group is bonded to silicon.

The alkyl group, the alkenyl group, the alkynyl group, the alkoxy group, the aryl group, the aryloxy group, the aryl amino group, the alkylamino group, the heteroaryl group, the alkylsilyl group, or the arylsilyl group of Formula 1 may be optionally substituted with one or more substituents. Substituents to be substituted at this time may include, for example, deuterium, boron, halogen, the hydroxyl group, the nitro group, the phosphoryl group, the alkyl group, the alkenyl group, the alkynyl group, the heteroalkyl group, the aryl group, the arylalkyl group, the heteroaryl group, the heteroarylalkyl group, the alkoxy group, the alkylamino group, the arylamino group, the heteroarylamino group, the alkylsilyl group, the arylsilyl group, and the aryloxy group, but are not limited to.

More specifically, in Formula 1, R₁ to R₂₀ may be hydrogen, deuterium, the alkyl group, the alkenyl group, the alkynyl group, the alkyl group substituted with deuterium, the alkenyl group substituted with deuterium, or the alkynyl group substituted with deuterium, but is not limited to. These substituents may be the same as described above for R₁ to R₂₈.

In addition, in Formula 1, any substituents pair of R₁ to R₂₈ (for example, substituents adjacent to each other) may be combined with each other to form a condensed ring structure of aliphatic, aromatic, aliphatic hetero, or aromatic hetero. The pair of adjacent substituents may refer to a pair of any one substituent and a substituent substituted on an atom directly connected to the atom where the substituent is substituted, a pair of any one substituent and a substituent that is sterically closest to the substituent, or any a pair of any one substituent and another substituent substituted for the atom where the substituent is substituted. For example, two substituents substituted at ortho positions in a benzene ring or two substituents substituted on the same carbon in an aliphatic ring may correspond to groups adjacent to each other.

In addition, since Formula 1 includes an absorption edge, it can have a structure capable of absorbing light of a desired wavelength. For example, the absorption edge may be a frame or structure having a so-called resonance structure and/or conjugated bond.

The optical absorption of light by compounds, especially organic compounds, is due to the energy difference (ΔE) between the ground state and the excited state. It is also explained by the energy difference of HOMO (Highest Unoccupied Molecular Orbital) and LUMO (Lowest Unoccupied Molecular Orbital).

In general, organic absorbents may include a resonance structure and/or conjugate bond as an absorption edge that can exhibit a light absorption effect. Accordingly, the compound may form a frame that allows to exhibit desired light absorption characteristics in general.

The specific types of the above-mentioned absorption edge or the frame is not particularly limited. As it is publicly known, the resonance effect refers to the interaction between a lone electron pair of a molecule and an adjacent π bonded electron pair, and the substituents or frames that cause such resonance effect are publicly known. In addition, a conjugated bond is a system composed of two or more double bonds with one single bond in-between and it is known that as the length of this conjugated bond increases, the energy difference between HOMO and LUMO decreases thereby causing the absorption band to shift to the long wavelength side.

For example, the absorption edge may be a frame or structure that allows the compound disclosed in this specification to exhibit an absorption maximum within a wavelength range of 690 nm to 1,200 nm.

The compounds disclosed in this specification may exhibit an absorption maximum wavelength within the range of 690 nm to 1,200 nm. In other examples, the lower limit of the absorption maximum wavelength may be about 690 nm, 700 nm, 710 nm, 720 nm, 730 nm, 740 nm, 750 nm, 760 nm, 770 nm, 780 nm, 790 nm, 810 nm, 820 nm, 830 nm, 840 nm, 850 nm, 860 nm, 870 nm, 880 nm, 890 nm, 900 nm, 910 nm or 920 nm. Additionally, the upper limit of the absorption maximum wavelength may be about 1,200 nm, 1,190 nm, 1,180 nm, 1,170 nm, 1,160 nm, 1,150 nm, 1,140 nm, 1,130 nm, 1,120 nm, or 1,100 nm. The absorption maximum wavelength may be within a range that is equal to and longer or longer than any one of the lower limits described above; it may be within a range that is equal to and longer or longer than any one of the above-described lower limits and equal to and shorter or shorter than any one of the above-described upper limits.

As mentioned above, the resonance structure and conjugation bond are determined by the energy difference (ΔE) between the ground state and excited state of the compound or the energy difference between HOMO (Highest Unoccupied Molecular Orbital) and LUMO (Lowest Unoccupied Molecular Orbital). Since the energy difference is determined and the absorption maximum wavelength is determined by this energy difference, the structure of the absorption edge can be determined so that the compound can have the absorption maximum wavelength in the range described above.

The compounds disclosed in the present specification may include deuterium for at least one of R₁ to R₂₈ in Formula 1. Here, “including deuterium” means to include a case where at least one of R₁ to R₂₈ is deuterium; at least one of R₁ to R₂₈ is the alkyl group where at least one of the hydrogen consisted of substituted with deuterium; at least one of R₁ to R₂₈ is the alkenyl group where at least one of the hydrogen consisted of substituted with deuterium; at least one of R₁ to R₂₈ is the alkynyl group where at least one of the hydrogen consisted of substituted with deuterium; at least one of R₁ to R₂₈ is the alkoxy group where at least one of the hydrogen consisted of substituted with deuterium; at least one of R₁ to R₂₈ is the aryl group where at least one of the hydrogen consisted of substituted with deuterium; at least one of R₁ to R₂₈ is the aryloxy group where at least one of the hydrogen consisted of substituted with deuterium; at least one of R₁ to R₂₈ is the arylamino group where at least one of the hydrogen consisted of substituted with deuterium; at least one of R₁ to R₂₈ is the alkylamino group where at least one of the hydrogen consisted of substituted with deuterium; at least one of R₁ to R₂₈ is the heteroaryl group where at least one of the hydrogen consisted of substituted with deuterium; at least one of R₁ to R₂₈ is the alkylsilyl group where at least one of the hydrogen consisted of substituted with deuterium; or at least one of R₁ to R₂₈ is the arylsilyl group where at least one of the hydrogen consisted of substituted with deuterium.

The compound represented by Formula 1 can secure excellent heat resistance without affecting the light absorption characteristics of the compound due to the deuterium contained in R₁ to R₂₈. More specifically, the compound represented by Formula 1 can secure excellent heat resistance due to the deuterium contained in R₁ to R₂₀.

The lower limits of the number of deuterium included in R₁ to R₂₈ of Formula 1 can be 1, 2, 3, 4, 5, 6, 7, 8, 9, 10, 11, 12, 13, 14, 15, 16, 17, 18, 19 or 20, the upper limits of the number of deuterium included in R₁ to R₂₈ of Formula 1 can be 200, 100, 90, 80, 70, 60, 50, 40, 30, 20, 19, 18, 17, 16, 15, 10, 9, 8, 7, 6, 5 or 4. The number of deuterium may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The deuterium substitution rate of the compounds disclosed in the present specification may be above a certain level. The deuterium substitution rate is theoretically the ratio of the number of moles of deuterium after deuterium substitution occurred based on the number of moles of all hydrogen contained in one mole of the compound before deuterium substitution occurred. The lower limits of the deuterium substitution rate may be about 10%, 15%, 20%, 25%, 30%, 40%, 45%, 50%, 55%, 60%, 65%, 70%, 75%, 80%, 85%, 90%, 95%, or 100%, and the upper limits may be about 100%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25% or 20%. The ratio may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits. Additionally, the deuterium substitution rate may mean how many hydrogen atoms directly bonded to carbon atoms are substituted with deuterium atoms. The method of measuring such deuterium substitution rate is described in ‘5. Deuterium Substitution Rate’ of Embodiments of the present specification.

The compounds disclosed in this specification can ensure heat resistance by controlling the deuterium substitution rate. The bond between carbon and deuterium has lower stretching and bending energies than the bond between carbon and hydrogen. Therefore, it is understood that the bond between carbon and deuterium can reduce the intramolecular vibrational energy compared to the bond between carbon and hydrogen thereby ensuring heat resistance by maintaining light absorption characteristics even in a high temperature and high humidity environment.

The compound may have an appropriate level of molar mass. For example, the lower limits of the molar mass may be about 900 g/mol, 950 g/mol, 1,000 g/mol, 1,100 g/mol, 1,150 g/mol, 1,200 g/mol, 1,250 g/mol, 1,300 g/mol, 1,350 g/mol, 1,400 g/mol, 1,450 g/mol, 1,460 g/mol, 1,470 g/mol, 1,480 g/mol, 1,490 g/mol, or 1,500 g/mol. The upper limits of the molar mass may be about 3,000 g/mol, 2,900 g/mol, 2,800 g/mol, 2,700 g/mol, 2,600 g/mol, 2,500 g/mol, 2,400 g/mol, 2,300 g/mol, 2,200 g/mol, 2,100 g/mol, 2,000 g/mol, 1,900 g/mol, 1,800 g/mol, 1,700 g/mol, 1,600 g/mol, 1,500 g/mol, or 1,490 g/mol. The molar mass may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The compound may have excellent heat resistance. For example, the 5% thermal decomposition temperature (hereinafter “Td 5%”) of the compound may be within a predetermined range. For example, the lower limits of Td 5% of the above compound may be about 285° C., 286° C., 287° C., 288° C., 289° C., 290° C., 291° C., 292° C., 293° C., 294° C., 295° C., 296° C., 297° C., 298° C., 299° C., or 300° C. The upper limits of Td 5% may be about 500° C., 480° C., 460° C., 440° C., 420° C., 400° C., 380° C., 360° C., 350° C., 340° C., 330° C., 320° C., 310° C., 300° C., or 290° C. The Td 5% may be within a range that is equal to and higher than or higher than any one of the lower limits described above; within a range that is equal to and lower or lower than any one of the above-described upper limits; or within a range that is equal to and higher than or higher than any one of the lower limits described above and is equal to and lower or lower than any one of the above-described upper limits.

The Td 5% is the temperature where mass loss of 95% occurs in Thermogravimetric Analysis (TGA) of the compound. This Td 5% is obtained through Thermogravimetric Analysis (TGA) and the Thermogravimetric Analysis (TGA) method is described in ‘3. Thermal Decomposition Temperature (Td 5%) Analysis’ of Embodiments of the present specification.

The compound may contain an anion. Examples of the anion may include halogen anion, hexafluoroantimonic acid anion (SbF⁶⁻), perchlorate anion, thiocyanate anion (SCN⁻), hexafluorophosphate anion (PF⁶⁻), phosphate anion, bis(trifluoromethanesulfonyl) imide anion, tetrafluoroboric acid anion (BF⁴⁻), trifluoro methylcarboxylic acid anion, alkylsulfonic acid anion, benzenesulfonic acid anion, toluenesulfonic acid anion, benzenecarboxylic acid anion, alkylcarboxylic acid anion, periodic acid anion, hydrofluoro borate anion and tetraphenylboricacid anion, but are not limited to.

The above compounds can be obtained through known synthesis methods of organic compounds and deuterium substitution methods.

The structure of Formula 1 is that of an absorbent known as a so-called dimmonium salt-based compound. Various methods for producing dimmonium salt-based compounds are known in the industry. Accordingly, for example, the compound of Formula 1 substitutes the reactant used in the production process of the known dimmonium salt-based absorbent with deuterium and it can be manufactured by applying this reactant to the absorbent synthesis process. Additionally, for example, the compound may be prepared by synthesizing a dimmonium salt-based absorbent according to a known synthesis method and then substituting at least part or all of the hydrogen in the synthesized absorbent with deuterium.

There is no particular limitation to the deuterium substitution method described above. For example, a method of mixing deuterium with a compound to be substituted with deuterium at an appropriate temperature may be applied. The lower limits of the mixing temperature may be, for example, about 90° C., 95° C., 100° C., 105° C., 110° C., 115° C., or 120° C., and the upper limits may be about 300° C., 280° C., 260° C., 240° C., 220° C., 200° C., 180° C., 160° C., 140° C., or 120° C. The mixing temperature may be within a range that is equal to and higher than or higher than any one of the lower limits described above; within a range that is equal to and lower or lower than any one of the above-described upper limits; or within a range that is equal to and higher than or higher than any one of the lower limits described above and is equal to and lower or lower than any one of the above-described upper limits.

The mixing time may not be particularly limited and, for example, it can be adjusted by taking into account the desired substitution ratio, etc. For example, the lower limits of the mixing time may be about 3 hours, 6 hours, 9 hours, 12 hours, 15 hours, 18 hours, 21 hours, or 24 hours, and the upper limits may be about 72 hours, 36 hours, or 24 hours. The mixing time may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

In addition, the mixing may be carried out in the presence of additional compounds capable of, for example, assisting, promoting or initiating the substitution with deuterium. For example, compounds that act as catalysts for substitution with deuterium may include any one of silver oxide (Ag₂O), silver acetate (AgOAc), silver trifluoroacetate (CF₃COOAg), silver carbonate (Ag₂CO₃), and palladium acetate (Pd(OAc)₂), palladium chloride (PdCl₂), bis(acetonitrile)dichloropalladium (PdCl₂(CH₃CN)₂), tris (dibenzylideneacetone)dipalladium (Pd₂(dba)₃), tetrakis(triphenylphosphine)palladium (Pd(PPh₃)₄) and bis(dibenzylideneacetone)palladium (Pd(dba)₂), but is not particularly limited as long as a compound that can act as a catalyst during deuterium substitution. In addition, compounds that act as ligands during deuterium substitution may include, for example, any one of allyldiphenylphosphine, allyldiphenylphosphine oxide, benzyldiphenylphosphine, 1-[2-[Bis(tert-butyl)phosphino]phenyl]-3,5-diphenyl-1H-pyrazole, bis[2-(diadamantylphosphino)ethyl]amine, bis(5H-dibenzo[a,d]cyclohepten-5-yl)phenylphosphine, bis(5H-dibenzo[a,d]cyclohepten-5-yl)phenylphosphine, 2-[bis(3,5-di-tert-butyl-4-methoxyphenyl) phosphino]benzaldehyde, 2,6-bis(di-tert-butylphosphinomethyl)pyridine, bis(dicyclohexylphosphinophenyl) ether, bis(diethylamino)phenylphosphine, 1,3-Bis-(2,6-diisopropylphenyl)-[1,3,2]diazaphospholidine 2-oxide, bis(dimethylamino) chlorophosphine, 2-[bis(3,5-dimethylphenyl) phosphino]benzaldehyde, 2,2′-bis(diphenylphosphino)-1,1′-biphenyl, bis(4-fluorophenyl) phenylphosphine oxide, bis[4-(3,3,4,4,5,5,5-heptafluoro-2,2-bis(trifluoromethyl)pentyl)phenyl]phenylphosphine, 1,1′-bis(phenylphosphinidene)ferrocene, (2-bromophenyl) dicyclohexylphosphine, (2-bromophenyl) diphenylphosphine, tert-butyldicyclohexylphosphine, tert-butyldiisopropylphosphine, tert-butyldiphenylphosphine, di-tert-butyl(2,2-diphenyl-1-methyl-1-cyclopropyl)phosphine, 2-chloro-1,3-bis(2,6-diisopropylphenyl)-1,3,2-diazaphospholidine, 2-dicyclohexylphosphino-2′,6′-bis(N,N-dimethylamino)biphenyl, 1-(dicyclohexylphosphino)-2,2-diphenyl-1-methylcyclopropane, dicyclohexyl(2,2-diphenyl-1-methyl-1-cyclopropyl)phosphine, cyclohexyldiphenylphosphine, 2-(dicyclohexylphosphino)-1,1-diphenyl-1-propene, dicyclohexyl(1-methyl-2,2-diphenylvinyl)phosphine, di(1-adamantyl)-2-dimethylaminophenylphosphine, di-1-adamantylphosphine, di(1-adamantyl)-(2-triisopropylsiloxyphenyl)phosphine, (5H-dibenzo[a,d]cyclohepten-5-yl)diphenylphosphine, (R)-(−)-1-[(S)-2-(di(3,5-bis-trifluoromethylphenylphosphino)ferrocenyl]ethyldicyclohexylphosphine, (R)-(−)-1-[(S)-2-(di(3,5-bis-trifluoromethylphenyl)phosphino)ferrocenyl]ethyldi(3,5-dimethylphenyl)phosphine, P,P-dichloroferrocenylphosphine, (R)-(−)-N,N-dimethyl-1-[(S)-2-(diphenylphosphino)ferrocenyl]ethylamine, and 1,2,3,4,5-Pentaphenyl-1′-(di-tert-butylphosphino) ferrocenes, but is not limited to as long as it is the compounds that can act as ligands during deuterium substitution.

The present specification also discloses compositions comprising the compounds. The term “composition” may refer to a mixture containing a compound and other ingredients or a mixture containing two or more types of compounds.

A composition containing such a compound basically includes the compound of Formula 1 above and may additionally contain other necessary components.

For example, the composition may further include a resin component that acts as a binder. There is no particular limitation on the type of the resin component applied in this case, and known resin components used to form a resin film, for example, a near-infrared resin film, can be applied. In the present specification, the compound component may exhibit appropriate compatibility or solubility with the various known resin components.

Examples of resin components may include one or more kinds selected from cyclic olefin (COP)-based resin, polyarylate resin, polyester resin, polysulfone resin, polyether sulfone resin, polyparaphenylene resin, polyarylene ether phosphine oxide resin, polyimide resin, polyetherimide resin, polyamidoimide resin, acrylic resin, polycarbonate resin, polyethylene naphthalate resin, or silicone resin, or various other organic resins or organic-inorganic hybrid resins, but it is not limited to.

Although not particularly limited, the compound of the present specification can be mixed with a cyclic olefin-based resin among the resin components that serve as a known binder to form a resin film showing excellent performance. Therefore, in one example, the resin component may be a cyclic olefin-based resin.

When the resin component is applied, there is no particular limitation to its ratio. For example, the resin component may be present such that the weight ratio of the compound to 100 parts by weight of the resin component ranges from 0.001 parts by weight to 10 parts by weight. The lower limits of the weight ratio of the compound to 100 parts by weight of the resin component may be, in other examples, about 0.001 parts by weight, 0.005 parts by weight, 0.01 parts by weight, 0.05 parts by weight, 0.1 parts by weight, 0.5 parts by weight, 1 parts by weight, 1.1 parts by weight, 1.2 parts by weight, 1.3 parts by weight, or 1.4 parts by weight and the upper limit may be about 10 parts by weight, 9 parts by weight, 8 parts by weight, 7 parts by weight, 6 parts by weight, 5 parts by weight, 4 parts by weight, 3 parts by weight, 2 parts by weight, or 1.5 parts by weight. The ratio may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

For example, the composition may further include a solvent in which the compound and/or the resin component is dispersed. There is no particular limitation on the type of solvent applied in this case and any known solvent used to form a resin film, for example, a near-infrared resin film, can be applied. In the present specification, the compound component may exhibit appropriate compatibility or solubility in various known solvents.

Examples of solvents may include methylene chloride, cyclohexanone, toluene, methyl ethyl ketone, methyl isobutyl ketone, propylene glycol methyl ether acetate, diethylene glycol monoethyl ether 3-methoxy butanol, ethylene glycol monobutyl ether acetate, 4-hydroxy-4-methyl-2-pentanone, gammabutyrolactone, pyridone, chloroform, 1,4-dioxane, ortho-dichlorobenzene, chlorobenzene, aliphatic alcohols having 2 or more carbon numbers (e.g., isobutyl alcohol, isopropyl alcohol, ethanol, isopropanol, butanol, etc.), butyl acetate, tetrahydrofuran, or xylene, but are not limited to.

When a solvent is applied, there is no particular limitation on the ratio, and the ratio can be adjusted within a range that allows appropriate dispersion of the compound and/or resin component.

The composition may contain any other necessary ingredients in addition to the ingredients described above. The optional ingredients may include, for example, adhesion imparting agents, leveling agents, antistatic agents, heat stabilizers, light stabilizers, antioxidants, dispersants, flame retardants, lubricants, or plasticizers, but are not limited to.

The present specification also relates to the use of the compounds or the compositions.

For example, the present specification may be about a resin film where the above compound or composition is applied. The resin film may contain at least a resin component and the above-mentioned compound. In this case, the specific types of the resin component and the ratio of the resin component to the compound are as described in the section for the compound composition.

The resin film may be a film capable of absorbing light within a predetermined range of wavelengths. In one example, the resin film may be an infrared resin film or a near-infrared resin film. For example, such a resin film may exhibit absorption characteristics in at least a portion of the wavelength range within the range of about 690 nm to 1,200 nm.

For example, the resin film may exhibit an absorption maximum wavelength within the range of 690 nm to 1,200 nm. In other examples, the lower limits of the absorption maximum wavelength may be about 690 nm, 700 nm, 710 nm, 720 nm, 730 nm, 740 nm, 750 nm, 760 nm, 770 nm, 780 nm, 790 nm, 810 nm, 820 nm, 830 nm, 840 nm, 850 nm, 860 nm, 870 nm, 880 nm, 890 nm, 900 nm, 910 nm, or 920 nm. Additionally, the upper limits of the absorption maximum wavelength may be about 1,200 nm, 1,190 nm, 1,180 nm, 1,170 nm, 1,160 nm, 1,150 nm, 1,140 nm, 1,130 nm, 1,120 nm, or 1,100 nm. The absorption maximum wavelength may be within a range that is equal to and longer than or longer than any one of the lower limits described above; within a range that is equal to and shorter or shorter than any one of the above-described upper limits; or within a range that is equal to and longer than or longer than any one of the lower limits described above and is equal to and shorter or shorter than any one of the above-described upper limits.

Due to these characteristics, the resin film can be applied to various devices such as optical filters and infrared sensors. Thus, the resin film can provide excellent optical properties and physical properties such as excellent heat resistance for the devices.

For example, the transmittance at the absorption maximum of the resin film may be below a certain level. The upper limit may be about 50%, 49%, 48%, 47%, 46%, 45%, 44%, 43%, 42%, 41%, or 40%, and the lower limit may be about 0.1%, 1%, 10%, 15%, 20%, 25%, 30%, 35%, or 39%. The transmittance at the absorption maximum may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

For example, assuming that the transmittance at the absorption maximum wavelength of the resin film after being maintained at 85° C. and 85% relative humidity for 120 hours is ‘A_{f}’ and the transmittance at the absorption maximum wavelength of the resin film before being maintained at 85° C. and 85% relative humidity for 120 hours is ‘Aᵢ’, the upper limit of an absolute value of A_{f}−Aᵢ may be about 40%, 35%, 30%, 25%, 20%, 15%, 10%, or 5% and the lower limit may be about 0%, 5%, 10%, 15%, 20%, or 25%. The absolute value of A_{f}−Aᵢ may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

For example, in the resin film, the absolute value of AA in Equation 1 may be less than or equal to a predetermined value.

$$Δ A = 100 \times ( {A}_{f} - {A}_{i} ) / {A}_{i} . \qquad [ Equation 1 ]$$

In Equation 1, A_{f} is the transmittance at the absorption maximum wavelength of the resin film after being maintained at 85° C. and 85% relative humidity for 120 hours, and Aᵢ is the transmittance at the absorption maximum wavelength of the resin film before being maintained at 85° C. and 85% relative humidity for 120 hours. The absorption maximum wavelength exists within the wavelength range of 690 nm to 1,200 nm.

The upper limit of the absolute value of AA in Equation 1 may be about 80%, 70%, 60%, 50%, 45%, 40%, 35%, 30%, 25%, 20%, 15%, or 10% and the lower limit may be about 0%, 1%, 2%, 3%, 4%, 5%, 6%, 7%, 8%, 9%, 10%, 20%, 30%, 40%, 50%, 60%, or 70%. The absolute value of AA may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

In Equation 1, the upper limit of A_{f} may be approximately 80%, 75%, 70%, 65%, 60%, 55%, 50%, or 45% and the lower limit may be about 10%, 20%, 30%, 40%, 50%, 60%, or 65%. The A_{f} may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

In the resin film, the absolute value of A) in Equation 2 may be less than or equal to a predetermined value.

$$Δλ = 100 \times ( {λ}_{f} - {λ}_{i} ) / {λ}_{i} . \qquad [ Equation 2 ]$$

In Equation 2, λ_{f} is the absorption maximum wavelength of the resin film after being maintained at 85° C. and 85% relative humidity for 120 hours and λᵢ is the absorption maximum wavelength of the resin film before being maintained at 85° C. and 85% relative humidity for 120 hours. The absorption maximum wavelength exists within the wavelength range of 690 nm to 1,200 nm.

The upper limit of the absolute value of Δλ in Equation 2 may be about 10%, 9%, 8%, 7%, 6%, 5%, 4%, 3%, 2%, 1%, or 0.5% and the lower limit may be 0%. The absolute value of Δλ may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

In Equation 2, λ_{f} and λᵢ may each be in the range of 690 nm to 1,200 nm. In other examples, the lower limits of each of λ_{f} and λᵢ may be about 690 nm, 700 nm, 710 nm, 720 nm, 730 nm, 740 nm, 750 nm, 760 nm, 770 nm, 780 nm, 790 nm, 810 nm, 820 nm, 830 nm, 840 nm, 850 nm, 860 nm, 870 nm, 880 nm, 890 nm, 900 nm, 910 nm or 920 nm. Additionally, the upper limits of each of λ_{f} and λᵢ may be about 1,200 nm, 1,190 nm, 1,180 nm, 1,170 nm, 1,160 nm, 1,150 nm, 1,140 nm, 1,130 nm, 1,120 nm, or 1,100 nm. Each of the λ_{f} and λᵢ may be within a range that is equal to and longer than or longer than any one of the lower limits described above; within a range that is equal to and shorter or shorter than any one of the above-described upper limits; or within a range that is equal to and longer than or longer than any one of the lower limits described above and is equal to and shorter or shorter than any one of the above-described upper limits.

Through the absorption properties, the resin film can be applied to various devices such as optical filters and infrared sensors. Thus, the resin film can provide for the devices to efficiently achieve the desired properties.

In the present specification, the resin film can be formed in a publicly known manner as long as the compound or composition of the present specification is applied. For example, the resin film can be formed by coating the compound composition in an appropriate manner and, if necessary, performing a curing or drying process.

There is no particular limitation on the thickness of the resin film and the thickness can be adjusted considering the desired characteristics. In one example, the resin film may have a thickness in the range of approximately 0.5 to 20 m.

The present specification also relates to an optical filter. The optical filter may include a substrate and the resin film formed on one or both sides of the substrate.

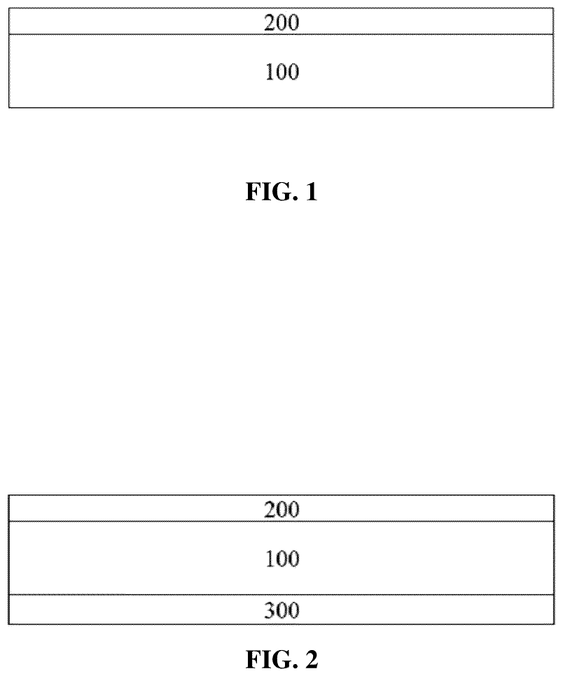

FIG. 1 is an example of the optical filter showing a case where the resin film 200 is formed on one surface of the substrate 100.

The optical filter of the present specification can exhibit excellent performance by including the above-described resin film. For example, the optical filter can efficiently and accurately block unnecessary infrared light and implement a visible light transmission band with high transmittance.

There is no particular limitation on the type of transparent substrate applied to the optical filter and publicly known transparent substrates for the optical filter can be used. In one example, the substrate may be a so-called near-infrared absorption substrate. A near-infrared absorption substrate is a substrate that exhibits absorption characteristics in at least a portion of the near-infrared region. A so-called blue glass, which exhibits the above-mentioned properties by including copper, is a representative example of the near-infrared absorption substrate. Such the near-infrared absorption substrate is useful in constructing an optical filter that blocks light in the near-infrared region, but due to the absorption characteristics, it is disadvantageous in terms of securing high transmittance in the visible light region and is also disadvantageous in terms of durability. In the present specification, an optical filter that efficiently blocks desired light, exhibits high transmittance characteristics in the visible light region, and has excellent durability can be provided by properly selecting a near-infrared absorption substrate and combining it with the specific resin film.

As for the infrared absorption substrate, a substrate showing an average transmittance above a certain level within the wavelength range of 425 nm to 560 nm can be used. The lower limit of the average transmittance may be about 75%, 77%, 79%, 81%, 83%, 85%, 87%, or 89%, and the upper limit may be about 98%, 96%, 94%, 92%, or 90%. The average transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

As for the infrared absorption substrate, a substrate exhibiting a maximum transmittance above a certain level within the wavelength range of 425 nm to 560 nm can be used. The lower limit of the maximum transmittance may be about 80%, 82%, 84%, 86%, 88%, or 90%, and the upper limit may be about 100%, 98%, 96%, 94%, 92%, or 90%. The maximum transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

As for the infrared absorption substrate, a substrate showing an average transmittance above a certain level within the wavelength range of 350 nm to 390 nm can be used. The lower limit of the average transmittance may be about 75%, 77%, 79%, 81%, or 83%, and the upper limit may be about 98%, 96%, 94%, 92%, 90%, 88%, 86%, or 84%. The average transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

As for the infrared absorption substrate, a substrate exhibiting a maximum transmittance above a certain level within the wavelength range of 350 nm to 390 nm can be used. The lower limit of the maximum transmittance may be about 80%, 82%, 84%, 86%, or 87%, and the upper limit may be about 100%, 98%, 96%, 94%, 92%, 90%, or 88%. The maximum transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

With the infrared absorption substrate, the transmittance at a wavelength of 700 nm may be within a certain range. The lower limit may be about 10%, 12%, 14%, 16%, 18%, 20%, 22%, 24%, 26%, or 28% and the upper limit may be about 45%, 43%, 41%, 39%, 37%, 35%, 33%, 31% or 29%. The transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The infrared absorption substrate may have an average transmittance within a wavelength range of 700 nm to 800 nm. The lower limit may be about 5%, 7%, 9%, 11%, 13%, 15%, 15.5%, 16%, or 16.5% and the upper limit may be 30%, 28%, 26%, 24%, 22%, 20%, 18% or 17%. The average transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

With the infrared absorption substrate, the maximum transmittance may be within a certain wavelength range of 700 nm to 800 nm. The lower limit may be about 10%, 12%, 14%, 16%, 18%, 20%, 22%, 24%, 26%, or 28% and the upper limit may be about 43%, 41%, 39%, 37%, 35%, 33%, 31%, or 29%. The maximum transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The infrared absorption substrate may have an average transmittance within a certain wavelength range of 800 nm to 1,000 nm. The lower limit may be about 3%, 5%, 7%, 9%, or 11% and the upper limit may be about 20%, 18%, 16%, 14%, or 12%. The average transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The infrared absorption substrate may have a maximum transmittance within a certain wavelength range of 800 nm to 1,000 nm. The lower limit may be about 5%, 7%, 9%, 11%, 13% or 15% and the upper limit may be about 30%, 28%, 26%, 24%, 22%, 20%, 18%, or 16%. The maximum transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The infrared absorption substrate may have an average transmittance within a certain wavelength range of 1,000 nm to 1,200 nm. The lower limit may be about 10%, 12%, 14%, 16%, 18%, 20%, 22%, 24%, or 25% and the upper limit may be about 50%, 48%, 46%, 44%, 42%, 40%, 38%, 36%, 34%, 32%, 30%, 28%, or 26%. The average transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The infrared absorption substrate may have a maximum transmittance within a certain wavelength range of 1,000 nm to 1,200 nm. The lower limit may be about 10%, 12%, 14%, 16%, 18%, 20%, 22%, 24%, 26%, 28%, 30%, 32%, 34%, or 36% and the upper limit may be about 70%, 68%, 66%, 64%, 62%, 60%, 58%, 56%, 54%, 52%, 50%, 48%, 46%, 44%, 42%, 40%, 38%, or 36%. The maximum transmittance may be within a range that is equal to and greater than or greater than any one of the lower limits described above; within a range that is equal to and less or less than any one of the above-described upper limits; or within a range that is equal to and greater than or greater than any one of the lower limits described above and is equal to and less or less than any one of the above-described upper limits.

The infrared absorption substrate can be combined with the resin film of the present specification to form a desired optical filter. As such a substrate, a substrate known as a so-called infrared absorbing glass can be used. Such glass is an absorption type glass manufactured by adding CuO or similar composition to a fluorophosphate-based glass or a phosphate-based glass. Accordingly, in an embodiment of the present specification, a CuO-containing fluorophosphate glass substrate or a CuO-containing phosphate glass substrate may be used as the infrared absorption substrate. The phosphate glass also includes K phosphate glass where a portion of the glass structure is composed of SiO₂. As such absorption-type glass is publicly known, the glass disclosed in Korean Patent No. 10-2056613 or other commercially available absorption-type glass (e.g., commercially available products from Hoya, Schott, PTOT, etc.), for example, can be used.

Such an infrared absorption substrate contains copper. In the present specification, a substrate having a copper content within the range of 1% by weight to 7% by weight may be used. In other examples, the copper content may be about 1.5% by weight or more, 2% by weight or more, 2.5% by weight or more, 2.6% by weight or more, 2.7% by weight or more, or 2.8% by weight or more or 6.5% by weight or less, 6% by weight or less, 5.5% by weight or less. It may be less than 5% by weight, less than 4.5% by weight, less than 4% by weight, less than 3.5% by weight, less than 3% by weight, or less than 2.9% by weight. A substrate having such the copper content is likely to exhibit the above-described optical properties and can be combined with the resin film to form an optical filter with desired properties.

The copper content can be confirmed by using X-ray fluorescence analysis equipment (WD XRF, Wavelength Dispersive X-Ray Fluorescence Spectrometry). When the X-ray is irradiated to a specimen (a substrate) from the equipment, characteristic secondary X-ray is generated from respective elements of the specimen and the equipment can detect the secondary X-ray with respect to the wavelength of each element. The intensity of the secondary X-rays is proportional to the element content, and thus, quantitative analysis can be performed through the intensity of the secondary X-ray.

The thickness of the infrared absorption substrate may be adjusted within a wavelength range of, for example, about 0.03 mm to 5 mm, but is not limited to.

The optical filter of the present specification may include other known components required in addition to the substrate and the resin film. For example, the optical filter may further include a dielectric film. For example, the dielectric layer may additionally include a so-called dielectric layer on one or both sides of the substrate.

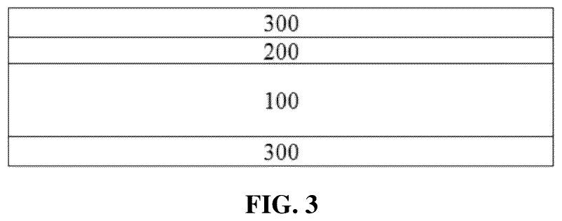

FIGS. 2 and 3 are examples of optical filters where a dielectric film 300 is added and illustrate a case where the dielectric film 300 is formed on one or both sides of a stacked structure including a substrate 100 and a resin film 200.

Such the dielectric film is a film composed of repeatedly stacking a dielectric material with a low refractive index and a dielectric material with a high refractive index. It is also used to form a so-called IR reflection layer and an anti-reflection (AR) layer. In the present specification, such a known IR reflection layer or a dielectric film for forming the AR layer may be applied.

Accordingly, the dielectric film may have a multi-layer structure including at least two types of sub-layers each having a different refractive index and may include a multi-layer structure where the two types of sub-layers are repeatedly stacked.

The material forming the dielectric film, in other words, the material forming each sub-layer is not particularly limited and publicly known material can be applied. In general, SiO₂ or fluorides such as Na₅Al₃Fl₄, Na₃AlF₆, or MgF₂ may be used to manufacture low-refractive sub-layers and amorphous silicon, TiO₂, Ta₂O₅, Nb₂O₅, ZnS, or ZnSe, etc. may be used to manufacture high-refractive sub-layers, but the material applied in the present specification is not limited to.

The method of forming the above-mentioned dielectric film is not particularly limited, and for example, it can be formed by applying a publicly known deposition method. In the industry, there is a known method of controlling the reflection or transmission characteristics of the dielectric film in consideration of the deposition thickness or number of layers of the sub-layer, and in the present specification, the dielectric film can be formed according to this known method.

Moreover, the optical filter may further include a resin film that exhibits absorption characteristics for ultraviolet ray (referred to as an ultraviolet ray resin film) as a resin film that is distinct from the resin film. However, this resin film may not be an essential component, and for example, an ultraviolet compound described later may be introduced to one resin film together with the compound of Formula 1.

In one example, the ultraviolet resin film may be designed to exhibit absorption maximum in a wavelength range of about 300 nm to 390 nm. The ultraviolet resin film may contain only the ultraviolet compound or, if necessary, may contain two or more types of ultraviolet compounds.

For example, as an ultraviolet compound, a publicly known compound that exhibits an absorption maximum in a wavelength range of about 300 nm to 390 nm can be used. The material and construction methods used to construct this ultraviolet resin film are not particularly limited and publicly known material and construction methods can be applied.

Usually, the ultraviolet resin film is formed by using material mixed with a transparent resin and an ultraviolet compound capable of exhibiting the desired absorption maximum. At this time, the transparent resin may be a resin component applied to the compound composition. In addition to the layers described above, the optical filter may further include various necessary layers within the scope of not impairing the desired effect.

The present specification also relates to an image capturing device including the optical filter. At this time, the configuration of the image capturing device or the application method of the optical filter is not particularly limited and publicly known configurations and application methods may be applied.

In addition, the use of the optical filter of the present specification is not limited to the image capturing device. It may be applied to various other applications requiring near-infrared ray cutting such as a display device including a PDP.

The present specification also relates to an infrared sensor including the resin film. The configuration of the infrared sensor is not particularly limited as long as it includes the resin film disclosed in the present specification. For example, it can be configured by introducing the resin film of the present specification into a publicly known motion sensor, proximity sensor, or gesture sensor.

In addition, the use of the compound composition or the resin film of the present specification is not limited to the optical filter, infrared sensor, and/or image capturing device. It can also be applied to various other applications that require infrared ray cutting such as an electronic component such as LiDAR.

The compounds disclosed in the present specification will be described in detail through Embodiments and Comparative Examples, but the scope of the compounds is not limited by Embodiments.

### 1. Absorption Maximum Measurement Method

The absorption maximum of the compound was evaluated by a conventional method. Specifically, the sample (compound) was dissolved in chloroform at a concentration of about 10⁻⁵ M and then, evaluated by using a measuring equipment (Agilent, Varian Cary 4000).

### 2. Evaluation of Transmittance Spectrum

The absorption maximum of the compound was measured by using a spectrophotometer (Perkinelmer, Lambda750 Spectrophotometer) from a specimen obtained by cutting the measurement object (e.g., a resin film) so that the width and height of the specimen was 10 mm and 10 mm, respectively. The transmittance spectrum was measured for each wavelength according to the equipment manual. The specimen was placed on a straight line between the measuring beam of the spectrophotometer and the detector and the transmittance spectrum was confirmed by setting the incident angle of the measuring beam to 0°. The incident angle of 0° is a direction substantially parallel to the normal direction of the surface of the specimen.

The average transmittance within a certain wavelength range in the transmittance spectrum is the result of measuring the transmittance at each wavelength while increasing the wavelength by 1 nm starting from the shortest wavelength in the wavelength range and then, calculating the arithmetic average of the measured transmittances. The maximum transmittance is the highest transmittance among the transmittances measured while increasing the wavelength by 1 nm and the minimum transmittance is the lowest transmittance among the transmittances measured while increasing the wavelength by 1 nm. For example, the average transmittance within the wavelength range of 350 nm to 360 nm is the arithmetic mean of the transmittance measured at the wavelength of 350 nm, 351 nm, 352 nm, 353 nm, 354 nm, 355 nm, 356 nm, 357 nm, 358 nm, 359 nm, and 360 nm. The maximum transmittance within the wavelength range of 350 nm to 360 nm is the highest transmittance among the transmittances measured at wavelengths of 350 nm, 351 nm, 352 nm, 353 nm, 354 nm, 355 nm, 356 nm, 357 nm, 358 nm, 359 nm, and 360 nm. The minimum transmittance within the wavelength range of 350 nm to 360 nm is the lowest transmittance among the transmittances measured at wavelengths of 350 nm, 351 nm, 352 nm, 353 nm, 354 nm, 355 nm, 356 nm, 357 nm, 358 nm, 359 nm, and 360 nm.

### 3. Pyrolysis Temperature (Td 5%) Analysis

Thermogravimetric Analysis (TGA) of the compound was performed by using Scinco's TGA N-1000 equipment. The analysis was performed by using approximately 3 mg of sample (compound) and the analysis was conducted under conditions of a temperature range of 25° C. to 800° C., a temperature increase rate of 10° C./min, and nitrogen (N₂) atmosphere of 60 cm³/min. The value at 95% weight loss (Td 5%) was used as the Td decomposition temperature.

### 4. Mass Analysis (MALDI-TOF)

Mass analysis of the compound was performed with a MALDI-TOF Voyager DE-STR equipment (Applied Biosystems, USA). It was measured with positive mode in reflector mode and analyzed using a dithranol matrix.

### 5. Deuterium Substitution Rate

Deuterium substitution rate of the compound was measured through hydrogen nuclear magnetic resonance (¹H NMR) analysis. ¹H-NMR analysis was performed on a compound substituted with deuterium (sample compound) and a compound with the same structure before substitution with deuterium (reference compound). The position and area of the hydrogen peak of the reference compound were confirmed through ¹H-NMR analysis of the reference compound and the position and area of the non-deuterium hydrogen peak and the deuterium peak of the sample compound were confirmed by performing ¹H-NMR analysis of the sample compound. The deuterium substitution rate was confirmed by comparing the peak positions and areas.

The substitution rate was calculated using Equation A.

$$Substitution rate = 100 \times D / H \qquad [ Equation A ]$$

In Equation A, D is the integral value of the deuterium peak in ¹H-NMR analysis for the sample compound. H is the integral value of the hydrogen peak in ¹H-NMR analysis for the reference compound.

Meanwhile, the ¹H-NMR analysis was performed by dissolving in CDCl₃ containing TMS using JEOL's JNM-ECX400. The chemical shift was expressed in ppm.

### Embodiment 1

Compound A of the Formula A below was synthesized in the following manner.

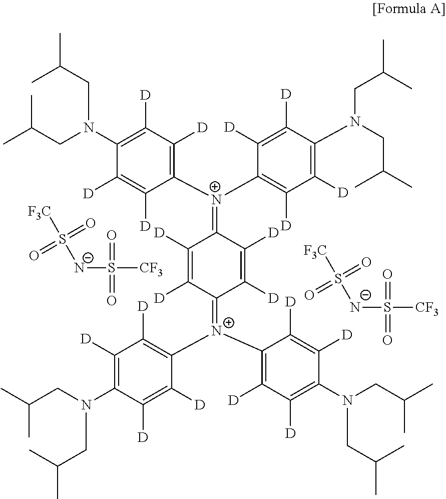

In Formula A, D is hydrogen or deuterium and at least one of D is deuterium. N,N,N,N-tetrakis(p-diisobutylamino phenyl)-p-phenylenedimonium-d20 (Compound A1) was prepared in a three-necked flask equipped with a reflux device by deuterium substitution of N,N,N,N-tetrakis (p-diisobutylaminophenyl)-p-phenylenedimonium (Compound D of Formula D of Comparative Example 1).

The deuterium-substituted Compound A1 was synthesized in the following manner. 9.21 g of Compound D of Comparative Example 1, 0.54 g of silver carbonate (Ag₂CO₃), and 1.34 g of cyclohexyldiphenylphosphine was added to a three-necked flask equipped with a reflux device. Then, 1 mL of toluene and an excessive deuterium oxide were added and the mixture was stirred at about 120° C. for 24 hours. 15 mL of dichloromethane and 15 mL of water were added to the flask and stirred and mixed for an additional 30 minutes. The dichloromethane layer was separated from the mixture by using a separatory funnel and methanol was added to precipitate the target compound (Compound A1) through recrystallization.

0.5 g of Compound A1, 0.7 g of lithium bistrifluoromethanesulfonnilimide, and 5 mL of dichloromethane was added to a three-necked flask and stirred. Then, 0.7 g of nitric acid and 1 mL of water was further added and stirred at room temperature (25° C.) for 2 hours. After stirring, dichloromethane and water was additionally added to the flask, the dichloromethane layer was separated by using a separatory funnel, and methanol was added to obtain the target compound (bistrifluoromethanesulfonylimide N,N,N,N-tetrakis (p-diisobutylaminophenyl)-p-phenylenedimonium-d20) (Compound A of Formula A) (MALDI-TOF m/z 1481.5 [M+H]⁺).

The deuterium substitution rate of the target Compound A was about 92%.

### Embodiment 2

Compound B of Formula B was synthesized in the following manner.

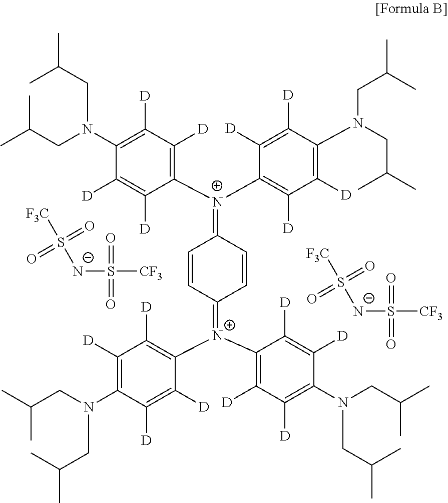

In Formula B, D is hydrogen or deuterium, but at least one of D is deuterium. 1-Iodo-4-nitrobenzene was substituted with deuterium. The deuterium substitution was performed according to the deuterium substitution method of Compound A1 of Embodiment 1, but the deuterium substitution was performed by using 2.49 g of 1-iodo-4-nitrobenzene instead of using 9.21 g of Compound D of Comparative Example 1.

2.0 g of deuterium-substituted 1-iodo-4-nitrobenzene, 0.2 g of 1,4-phenylenediamine, 0.04 g of copper powder, and 0.59 g of potassium carbonate was added to a three-necked flask and dimethylform (DMF) was added as a solvent. After adding the solvent, reaction was performed at room temperature (25° C.) under reflux condition for about 12 hours. Copper powder and potassium carbonate was removed through a filter to obtain N,N,N,N-tetrakis (4-nitrophenyl)-1,4-phenylenediamine. 1.5 g of N,N,N,N-tetrakis (4-nitrophenyl)-1,4-phenylenediamine and 0.5 g of tin chloride was added to the three-necked flask, 10 mL of HCl as a solvent was further added and then, the reaction was proceeded at room temperature (25° C.). After the reaction was proceeded sufficiently, 5 mL of methanol was added to the reaction solution to obtain N,N,N,N-tetrakis (4-aminophenyl)-1,4-phenylenediamine.

1 g of N,N,N,N-tetrakis (4-aminophenyl)-1,4-phenylenediamine, 4 g of isobutyl bromide, and 0.6 g of potassium carbonate was added to a three-necked flask and DMF as a solvent was added. Afterwards, the reaction was performed at room temperature (25° C.) under reflux condition for about 9 hours. After completion of the reaction, potassium carbonate was removed through a filter, the reaction solution was placed in a separatory funnel, 10 mL of dichloromethane and 10 mL of water was added, and then, the dichloromethane layer was separated. Methanol was added to the separated dichloromethane layer to obtain N,N,N,N-tetrakis (p-diisobutylaminophenyl)-1,4-phenylenediamine.

1 g of N,N,N,N-tetrakis (p-diisobutylaminophenyl)-p-phenylenediamine, 1.4 g of lithium bistrifluoromethanesulfonnilimide, and 20 mL of dichloromethane was added to a three-neck flask and then, stirred. 0.14 g of nitric acid and 2 mL of water was further added and the mixture was stirred for an additional 2 hours to react. After reaction, dichloromethane and water was additionally added to the flask, the dichloromethane layer was separated by using a separatory funnel, and methanol was added to obtain bistrifluoromethanesulfonylimide N,N,N,N-tetrakis (p-diisobutylaminophenyl)-p-phenylenedimonium-d16 (Compound B of Formula B) (MALDI-TOF m/z 1496.2 [M+H]⁺).

The deuterium substitution rate of Compound B was about 74%.

### Embodiment 3

Compound C of the Formula C below was synthesized in the following manner.

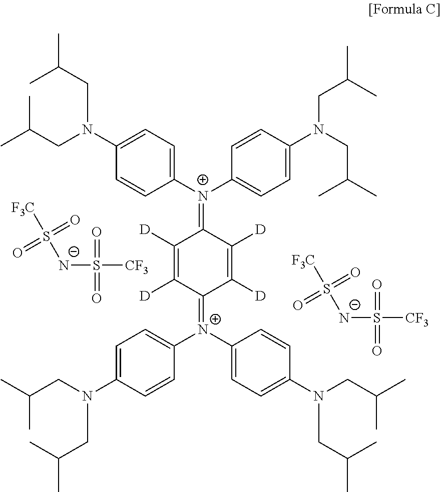

In Formula C, D is hydrogen or deuterium and at least one of D is deuterium. First, deuterium-substituted 1,4-phenylenediamine was synthesized. The deuterium substitution was performed according to the deuterium substitution method of Compound A1 of Embodiment 1, but the deuterium substitution was performed by using 1 g of 1,4-phenylenediamine instead of using 9.21 g of Compound D of Comparative Example 1.

2.0 g of 1-iodo-4-nitrobenzene, 0.2 g of 1,4-phenylenediamine substituted with long-term deuterium, 0.04 g of copper powder, and 0.59 g of potassium carbonate was added to a three-necked flask, dimethylform (DMF) was added as a solvent, and then, reaction was performed at room temperature (25° C.) under reflux condition for about 12 hours. Copper powder and potassium carbonate was removed through a filter to obtain N,N,N,N-tetrakis (4-nitrophenyl)-1,4-phenylenediamine. 1.5 g of N,N,N,N-tetrakis (4-nitrophenyl)-1,4-phenylenediamine and 0.5 g of tin chloride was to the three-necked flask, 10 mL of HCl was further added as a solvent, and then, the reaction was proceeded at room temperature (25° C.). After the reaction was proceeded sufficiently, 5 mL of methanol was added to the reaction solution to obtain N,N,N,N-tetrakis (4-aminophenyl)-1,4-phenylenediamine.

1 g of N,N,N,N-tetrakis (4-aminophenyl)-1,4-phenylenediamine, 4 g of isobutyl bromide, and 0.6 g of potassium carbonate was added to a three-necked flask and DMF was added as a solvent. Afterwards, the reaction was performed at room temperature (25° C.) under reflux condition for about 9 hours. After completion of the reaction, potassium carbonate was removed through a filter, the reaction solution was placed in a separatory funnel, 10 mL of dichloromethane and 10 mL of water was further added, and then, the dichloromethane layer was separated. Methanol was added to the separated dichloromethane layer to obtain N,N,N,N-tetrakis (p-diisobutylaminophenyl)-1,4-phenylenediamine.

1 g of N,N,N,N-tetrakis (p-diisobutylaminophenyl)-p-phenylenediamine, 1.4 g of lithium bistrifluoromethanesulfonnilimide, and 20 mL of dichloromethane was added to a three-neck flask and then stirred. After stirring, 0.14 g of nitric acid and 2 mL of water was further added, and then, the mixture was stirred for an additional 2 hours to react. After reaction, dichloromethane and water was additionally added to the flask, the dichloromethane layer was separated by using a separatory funnel, and methanol was added to obtain bistrifluoromethanesulfonylimide N,N,N,N-tetrakis (p-diisobutylaminophenyl)-p-phenylenediimonium-d4 (Compound C of Formula C) (MALDI-TOF m/z 1485.2 [M+H]+).

The deuterium substitution rate of Compound C was about 17%.

### Comparative Example 1

Compound D of Formula D below was synthesized in the following manner.

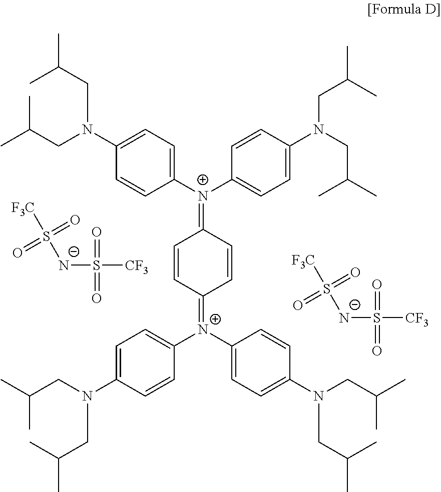 [SMILES: CC(C)CN(CC(C)C)c1ccc([N+](=C2C=CC(=[N+](c3ccc(N(CC(C)C)CC(C)C)cc3)c3ccc(N(CC(C)C)CC(C)C)cc3)C=C2)c2ccc(N(CC(C)C)CC(C)C)cc2)cc1 ; O=S(=O)([N-]S(=O)(=O)C(F)(F)F)C(F)(F)F ; O=S(=O)([N-]S(=O)(=O)C(F)(F)F)C(F)(F)F]

2.0 g of 1-iodo-4-nitrobenzene, 0.2 g of 1,4-phenylenediamine, 0.04 g of copper powder, and 0.59 g of potassium carbonate was added to a three-necked flask, dimethylformamide (DMF) was added as a solvent, and then, reacted at room temperature (25° C.) under reflux condition for about 12 hours. Copper powder and potassium carbonate was removed through a filter to obtain N,N,N,N-tetrakis (4-nitrophenyl)-1,4-phenylenediamine. 1.5 g of N,N,N,N-tetrakis (4-nitrophenyl)-1,4-phenylenediamine and 0.5 g of tin chloride was added to the three-necked flask, 10 mL of HCl was further as a solvent, and the reaction was proceeded at room temperature (25° C.). After the reaction was proceeded sufficiently, 5 mL of methanol was added to the reaction solution to obtain N,N,N,N-tetrakis (4-aminophenyl)-1,4-phenylenediamine.

1 g of N,N,N,N-tetrakis (4-aminophenyl)-1,4-phenylenediamine, 4 g of isobutyl bromide, and 0.6 g of potassium carbonate was added to a three-necked flask and then, DMF was added as a solvent. Afterwards, the reaction was performed at room temperature (25° C.) under reflux condition for about 9 hours. After completion of the reaction, potassium carbonate was removed through a filter, the reaction solution was placed in a separatory funnel, 10 mL of dichloromethane and 10 mL of water was added, and then, the dichloromethane layer was separated. Methanol was added to the separated dichloromethane layer to obtain N,N,N,N-tetrakis (p-diisobutylaminophenyl)-1,4-phenylenediamine.

1 g of N,N,N,N-tetrakis (p-diisobutylaminophenyl)-p-phenylenediamine, 1.4 g of lithium bistrifluoromethanesulfonnilimide, and 20 mL of dichloromethane was added to a three-neck flask and then, stirred. Then, 0.14 g of nitric acid and 2 mL of water was further added and the mixture was stirred for an additional 2 hours to react. After reaction, dichloromethane and water was additionally added to the flask, the dichloromethane layer was separated by using a separatory funnel, and methanol was added to obtain N,N,N,N-tetrakis (p-diisobutylaminophenyl)-p-phenylenediimonium (Compound D of Formula D) (MALDI-TOF m/z 1481.5 [M+H]⁺).

The deuterium substitution rate of Comparative Example 1 was approximately 0%.

Table 1 summarizes the absorption capacity of each Compound in Embodiments 1 to 3 and Comparative Example 1. In Table 1, T %(λₘₐₓ) is the transmittance at each absorption maximum wavelength. Td 5% in Table 1 is the temperature (Td 5%) where weight loss of 95% of the compound occurred in Thermogravimetric Analysis (TGA).

TABLE 1

| | Absorption Maximum | | |
|---|---|---|---|
| | Wavelength (nm) | T %(λₘₐₓ) | Td 5% (° C.) |
| Embodiment 1 | 1,100 | 39.2 | 300 |
| Embodiment 2 | 1,100 | 39.2 | 294 |
| Embodiment 3 | 1,100 | 39.5 | 289 |
| Comparative | 1,100 | 40.0 | 286 |
| Example 1 | | | |

Through Table 1, it can be confirmed that each Compound of Embodiments 1 to 3 exhibits light absorption characteristics equivalent to those of Comparative Example 1 while exhibiting excellent heat resistance.

### Test Example 1

A coating solution was prepared by mixing a cyclic olefin resin (TOPAS, 5013F-04), a compound, and a solvent (Cyclohexanone). Compounds synthesized in Embodiments or Comparative Examples were used as the compounds. The mixing ratio of the cyclic olefin-based resin, the compounds, and the solvent was about 69.3:0.99:29.7 (the weight ratio of cyclic olefin-based resin:compound:solvent). The coating solution was coated on a transparent substrate (a glass substrate manufactured by SCHOTT Co., Ltd.) and maintained at 140° C. for about 2 hours to form a resin film with a thickness of about 6 m.

Table 2 below summarizes the transmittance of the resin film before and after reliability evaluation in the visible and infrared regions. The reliability evaluation is an evaluation of maintaining the resin film at 85° C. under 85% relative humidity for 120 hours. In Table 2, ‘B’ refers to the result before performing the reliability evaluation and ‘A’ refers to the result after performing the reliability evaluation. Additionally, in Table 2, Λₘₐₓ refers to the transmittance at the absorption maximum (1,100 nm).

In Table 2, Δ is the change rate (%) of each characteristic before and after the reliability evaluation, which is the result calculated as 100×(A−B)/B where A is the value indicated by A in Table 2, and B is the value indicated by B in Table 2. In Table 2, T is the transmittance at the corresponding wavelength, Tₘᵢₙ refers to the minimum transmittance within the corresponding wavelength range, and Tₐᵥₑ refers to the average transmittance within the corresponding wavelength range.

TABLE 2

| Wavelength | | | | | | | | | | | | | |
|---|---|---|---|---|---|---|---|---|---|---|---|---|---|
| (nm) or | | | | | | | | | | | Comparative | | |
| Wavelength | | Embodiment 1 | | | Embodiment 2 | | | Embodiment 3 | | | Example 1 | | |
| Range (nm) | | B | A | Δ | B | A | Δ | B | A | Δ | B | A | Δ |
| 425~465 | Tₘᵢₙ | 88.8 | 88.1 | −0.8 | 89 | 89.3 | 0.3 | 88.9 | 81.7 | −8.1 | 88.4 | 82.7 | −6.4 |
| | Tₐᵥₑ | 89.1 | 88.6 | −0.6 | 89.2 | 89.6 | 0.4 | 89.2 | 83.5 | −6.4 | 88.9 | 84.2 | −5.3 |
| 466~480 | Tₘᵢₙ | 89.1 | 89 | −0.1 | 89.4 | 89.7 | 0.3 | 89.3 | 86.1 | −3.6 | 89 | 86.4 | −2.9 |
| | Tₐᵥₑ | 89.2 | 89.1 | −0.1 | 89.3 | 89.8 | 0.6 | 89.3 | 87.1 | −2.5 | 89.1 | 87.3 | −2.0 |
| 481~560 | Tₘᵢₙ | 89.3 | 89.3 | 0.0 | 89.2 | 89.9 | 0.8 | 89.3 | 88.1 | −1.3 | 89.2 | 88.4 | −0.9 |
| | Tₐᵥₑ | 89.8 | 89.9 | 0.1 | 89.8 | 90.3 | 0.6 | 89.8 | 90.5 | 0.8 | 89.8 | 90.7 | 1.0 |
| 561~630 | Tₘᵢₙ | 90 | 89.9 | −0.1 | 90 | 90.3 | 0.3 | 90 | 90.3 | 0.3 | 90.1 | 90.7 | 0.7 |
| | Tₐᵥₑ | 90.3 | 90.2 | −0.1 | 90.4 | 90.7 | 0.3 | 90.3 | 90 | −0.3 | 90.3 | 90.9 | 0.7 |
| 700 | T | 86.7 | 86.8 | 0.1 | 86.7 | 88.3 | 1.8 | 86.7 | 88.9 | 2.5 | 86.8 | 90.3 | 4.0 |
| 851~1,100 | Tₘᵢₙ | 39 | 42.5 | 9.0 | 39.1 | 51.4 | 31.5 | 39.5 | 67.9 | 71.9 | 39.9 | 81.3 | 103.8 |
| | Tₐᵥₑ | 49.4 | 51.6 | 4.5 | 49.6 | 60 | 21.0 | 49.7 | 72 | 44.9 | 50.1 | 83 | 65.7 |
| 950 | T | 47.5 | 50.1 | 5.5 | 47.1 | 58.4 | 24.0 | 47.4 | 70.8 | 49.4 | 48.1 | 83.2 | 73.0 |
| Λₘₐₓ | T | 39.2 | 42.5 | 8.4 | 39.2 | 51.4 | 31.1 | 39.5 | 67.9 | 71.9 | 40 | 84.5 | 111.3 |

### Test Example 2

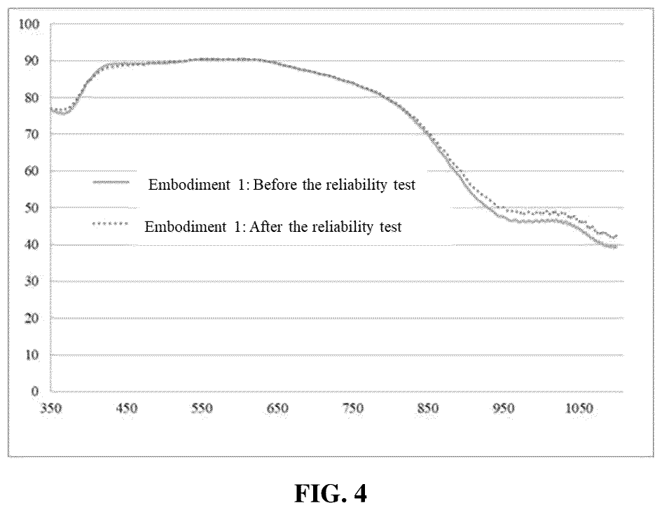

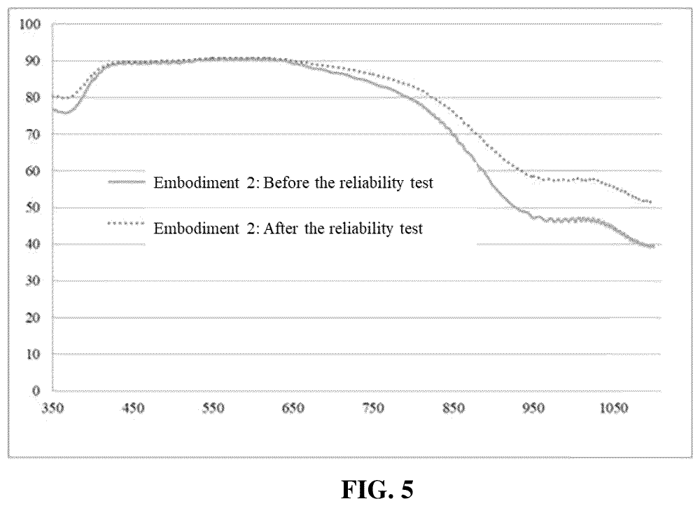

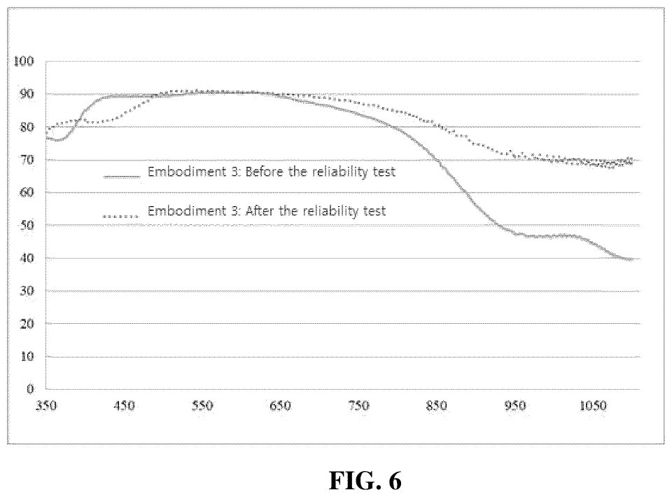

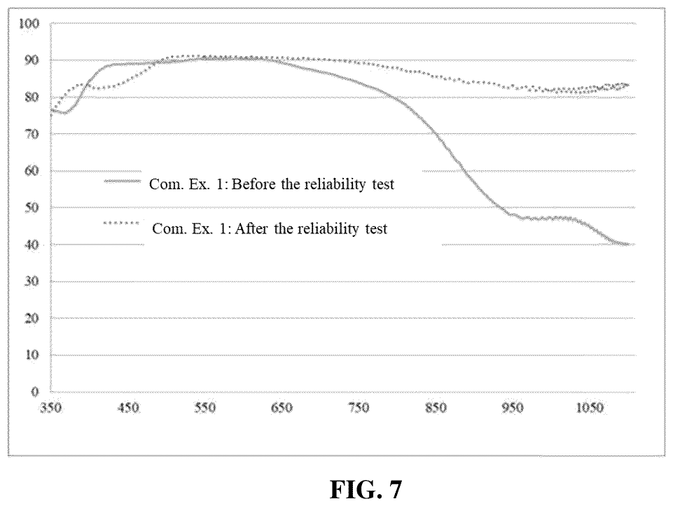

FIGS. 4 to 7 show evaluation results for the resin film prepared in the manner of Test Example 1 using Compounds of Embodiments 1 to 3 and Comparative Example 1, respectively. In FIGS. 4 to 7, the horizontal axis represents wavelength (nm), and the vertical axis represents transmittance (%). In addition, the results disclosed as ‘Before Reliability Test’ are the results immediately after manufacturing the resin film but before leaving the resin under the high temperature and high humidity condition and the results disclosed as ‘After Reliability Test’ are the results after performing a reliability evaluation (the same conditions as Test Example 1) on the resin film as leaving the resin film under the high temperature and high humidity condition.

Referring to FIGS. 4 to 7, the resin films using Compounds of Embodiments show little change in light absorption characteristics before and after high temperature and high humidity condition. In contrast, it can be confirmed that the resin film using Compound of Comparative Example had a very large change in light absorption characteristics before and after high temperature and high humidity condition and thus, the light absorption characteristics were almost lost.

The main contents of FIGS. 4 to 7 are summarized in Table 3 below. In Table 3, A_{f} is the transmittance at the absorption maximum wavelength of the resin film maintained at 85° C. under 85% relative humidity for 120 hours, λ_{f} is the absorption maximum wavelength at this time, and Aᵢ is the transmittance at the absorption maximum wavelength of the resin film before being maintained at 85° C. under 85% relative humidity for 120 hours and λᵢ is the absorption maximum wavelength at this time.

In Table 3, ΔA is a value calculated as 100×(A_{f}−Aᵢ)/Aᵢ and λₖ is a value calculated as 100×(λ_{f}−λᵢ)/λᵢ.

TABLE 3

| | | | | Comparative |
|---|---|---|---|---|
| | Embodiment 1 | Embodiment 2 | Embodiment 3 | Example 1 |
| Aᵢ | 39.2% | 39.20% | 39.5% | 40.0% |
| A_{f} | 42.5% | 51.4% | 67.9% | 84.50% |
| ΔA | 8.4 | 31.1 | 71.9 | 111.3 |
| λᵢ | 1,100 nm | 1,100 nm | 1,100 nm | 1,100 nm |
| λ_{f} | 1,100 nm | 1,100 nm | 1,100 nm | 1,100 nm |
| Δλ | 0% | 0% | 0% | 0% |

Comparing the results of Tables 1 to 3 and FIGS. 4 to 7, Compounds of Embodiments and Comparative Examples have similar spectral characteristics of the compounds themselves, but it can be confirmed that there is a big difference in absorption characteristics when applied to a resin film and/or absorption characteristics after evaluation at high temperature under high humidity. From this, it can be confirmed that the compound can absorb light in the infrared region due to its unique structure. Moreover, the compound can effectively form a resin film with excellent performance because it has excellent heat resistance at the same time.

1. A compound represented by Formula 1: 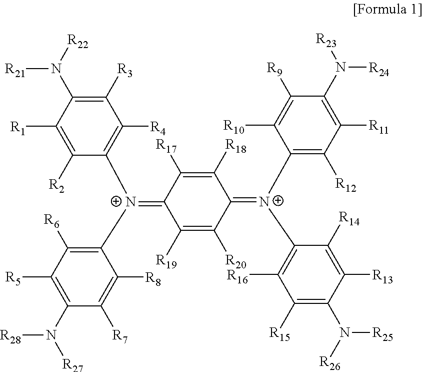
wherein
R₁ to R₂₈ are each independently hydrogen, deuterium, an alkyl group, an alkenyl group, an alkynyl group, an alkoxy group, an aryl group, an aryloxy group, an arylamino group, an alkylamino group, a heteroaryl group, an alkylsilyl group, an arylsilyl group, an amino group, a nitro group, a nitrile group, a hydroxy group, or cyano group; and at least one of R₁ to R₂₈ is deuterium.

2. The compound of claim 1, wherein a deuterium substitution rate of the compound is 10% or higher.

3. The compound of claim 1, wherein the compound exhibits an absorption maximum within a wavelength range of 690 nm to 1,200 nm.

4. The compound of claim 3, wherein a transmittance of the compound at the absorption maximum is 50% or less.

5. The compound of claim 1, wherein a molar mass of the compound is from 900 g/mol to 3,000 g/mol.

6. The compound of claim 1, wherein a 5% pyrolysis temperature of the compound is at least 285° C.

7. An ionic compound, comprising the compound of claim 1; and an anion.

8. A composition, comprising:
a resin component; and
the compound of claim 1.

9. The composition of claim 8, wherein the resin component comprises at least one or more selected from a group consisting of a cyclic olefin resin, a polyarylate resin, a polyester resin, a polysulfone resin, a polyether sulfone resin, a polyparaphenylene resin, a polyarylene ether phosphine oxide resin, a polyimide resin, a polyetherimide resin, a polyamidoimide resin, an acrylic resin, a polycarbonate resin, a polyethylene naphthalate resin, and a silicone resin.

10. The composition of claim 8, wherein the compound is comprised in an amount of 0.001 to 10 parts by weight, relative to 100 parts by weight of the resin component.

11. A resin film comprising:
a resin component; and
the compound of claim 1.

12. The resin film of claim 11, wherein the resin component comprises at least one or more selected from a group consisting of a cyclic olefin resin, a polyarylate resin, a polyester resin, a polysulfone resin, a polyether sulfone resin, a polyparaphenylene resin, a polyarylene ether phosphine oxide resin, a polyimide resin, a polyetherimide resin, a polyamidoimide resin, an acrylic resin, a polycarbonate resin, a polyethylene naphthalate resin, and a silicone resin.

13. The resin film of claim 11, wherein the resin component comprises a cyclic olefin resin.

14. The resin film of claim 11, wherein the resin film exhibits an absorption maximum within a wavelength range of 690 nm to 1,200 nm.

15. The resin film of claim 11, wherein an absolute value of AA of the Equation 1 below is 80% or less:
$$Δ A = 100 \times ( {A}_{f} - {A}_{i} ) / {A}_{i} \qquad [ Equation 1 ]$$
wherein A_{f} is a transmittance of the resin film at a wavelength of the absorption maximum of the resin film after maintaining the resin film at 85° C. and 85% relative humidity for 120 hours and Aᵢ is a transmittance of the resin film at a wavelength of the absorption maximum of the resin film before maintaining the resin film at 85° C. and 85% relative humidity for 120 hours.

16. The resin film of claim 11, wherein an absolute value of AX of the Equation 2 below is 10% or less:
$$Δλ = 100 \times ( {λ}_{f} - {λ}_{i} ) / {λ}_{i} \qquad [ Equation 2 ]$$
wherein
λ_{f} is a wavelength of the absorption maximum of the resin film after maintaining the resin film at 85° C. and 85% relative humidity for 120 hours and λᵢ is a wavelength of the absorption maximum of the resin film before maintaining the resin film at 85° C. and 85% relative humidity for 120 hours.

17. The resin film of claim 15, wherein the wavelength of the absorption maximum of the resin film exists within a wavelength range of 690 nm to 1,200 nm.

18. An optical filter, comprising:
a substrate; and
the resin film of claim 11 formed on one or both sides of the substrate.

19. An image capturing device comprising the optical filter of claim 18.

20. An infrared sensor comprising the resin film of claim 11.
